(19) United States
(12) Patent Application Publication
(10) Pub. No.: US 2019/0078152 A1
(43) Pub. Date: Mar. 14, 2019

(54) METHODS FOR DETECTING NUCLEOTIDE VARIANTS

(71) Applicant: VITO NV, Mol (BE)

(72) Inventors: Jef HOOYBERGHS, Mol (BE); Dirk VALKENBORG, Mol (BE)

(21) Appl. No.: 16/085,165

(22) PCT Filed: Mar. 17, 2017
(86) PCT No.: PCT/EP2017/056344

§ 371 Date: Sep. 14, 2018

(30) Foreign Application Priority Data
Mar. 17, 2016 (EP) 16160862.5

(51) Int. Cl. C12Q 1/6837; C12Q 1/6876

(52) U.S. Cl. CPC C12Q 1/6837; G06F 19/20; G06F 19/22; C12Q 1/6876

(57) ABSTRACT

Provided herein are methods for determining the presence of a mutant of a target wild type polynucleotide in a sample solution, comprising contacting and hybridizing the sample solution with the first and second probe of a well-chosen probe pair, and comparing the measured hybridisation intensities. Particularly, the probe pair is designed such that a first probe is specific for the mutant polynucleotide and the second probe is fully complementary to the wild type polynucleotide except for one or more nucleotides (different from the nucleotide differing between mutant and wild type polynucleotides).

### FIELD OF THE INVENTION

The present invention relates to methods for analyzing hybridisation, more particularly to determine the presence or absence of specific polynucleotide variants or mutants in a sample by analysing the hybridisation of the polynucleotide variants or mutants with specific probes.

### BACKGROUND OF THE INVENTION

In diagnostic or prognostic tests, one is often confronted with samples of mixed genomic sequence variants, some possibly present in minority. Nevertheless, an accurate identification of reliable mutation markers is crucial for allowing accurate molecular subtyping of disease and the rational use of molecularly guided therapies e.g. in cancer. The heterogeneity of the patient sample often complicates the detection and/or identification of such mutations. Indeed, biopsies such as from cancer tissue usually contain a mixture of cancerous and non-cancerous cells, with varying ratio of tumor vs non-tumor cells and wherein the mutations may be heterozygous or homozygous and possibly change over time.

In practice, the detection of DNA mutations is typically performed using polymerase chain reaction (PCR) and sequencing. The use of hybridisation techniques like microarrays for the detection of DNA mutations is rather uncommon, even though this technology is mature, affordable, widely used and very flexible towards type and number of DNA sequences to be analyzed. A remaining challenge with hybridisation techniques however is obtaining a clear quantitative interpretation of the microarray data. WO2011/035801 describes a method for analyzing hybridisation, involving the analysis of hybridisation intensities for different probes as a function of hybridisation free energy. Hooyberghs et al. (Biosensors and Bioelectronics, 2010, 26: 1692-1694) relates to a microarray and hybridisation-based method of detecting small concentrations in a mixture of mutant and wild type polynucleotides, based on the observation of a shift of cluster of probes with respect to a thermodynamic baseline (by plotting the hybridisation intensity against the ΔΔG), wherein hybridisation intensities obtained for the mixture are compared to this thermodynamic baseline. However, this and other hybridisation-based methods are prone to errors due to concentration variations in the sample.

Accordingly, there is still a need for improved methods for analyzing hybridisation, in particular for the reliable identification and/or quantification of mutant polynucleotides in a sample.

### SUMMARY OF THE INVENTION

The present invention relates to methods for analyzing hybridisation. More particularly, the methods described herein allow for the detection of the presence of specific polynucleotide variants such as mutant genes in a sample, particularly in a sample comprising both a target, wild type polynucleotide T_{wt} and a variant T_{M} thereof, and allow a reliable identification and/or quantification of such variant polynucleotides in a sample with a minimal number of probes and more particularly without the need for analysing a reference sample.

A first aspect of the present invention provides a method for determining the presence of a mutant polynucleotide T_{M} in a sample solution, particularly a sample solution comprising a target polynucleotide T_{wt} and said mutant polynucleotide T_{M}, said mutant polynucleotide T_{M} comprising a mutant sequence and differing from a target polynucleotide T_{wt} comprising a target sequence in one or more nucleotides of said target sequence, said method comprising:
(i) providing a probe pair comprising a first probe P_{A} and a second different probe P_{B}, wherein
   the hybridisation sequences of said first probe P_{A} and second probe P_{B} are characterized in that they each comprise at least one non-complementary nucleotide with respect to the target sequence of T_{wt}, particularly characterized in that the first probe P_{A} comprises at least one non-complementary nucleotide (NT1) with respect to the target sequence of T_{wt} and the second probe P_{B} comprises at least one different non-complementary nucleotide (NT2) with respect to the target sequence of T_{wt};
   the hybridisation sequence of said first probe P_{A} comprising one or more nucleotides complementary to the one or more nucleotides of the mutant sequence of T_{M} differing from said target sequence; and
   wherein the ratio of the hybridisation intensity [I(P_{A})] for the hybridisation between the target polynucleotide T_{wt} and the first probe P_{A} to the hybridisation intensity [I(P_{B})] for the hybridisation between the target polynucleotide T_{wt} and the second probe P_{B} is a known value;
(ii) contacting said sample solution with said first probe P_{A} and second probe P_{B} of said probe pair, and obtaining a first and second measured hybridisation intensity I(P_{A})m and I(P_{B})m for said first and second probe, respectively; and
(iii) analysing and comparing I(P_{A})m and I(P_{B})m and determining the presence of said mutant polynucleotide T_{M} in said sample solution based thereon.

In particular embodiments, said first probe P_{A} and second probe P_{B} are selected so that the ratio of the hybridisation intensity [I(P_{A})] for the hybridisation between the target polynucleotide and the first probe P_{A} to the hybridisation intensity [I(P_{B})] for the hybridisation between the target polynucleotide T_{wt} and the second probe P_{B} is known and ranges between 0.02 and 50, preferably between 0.1 and 10. In particular embodiments the ratio I(P_{A})/I(P_{B}) is about 1.

In particular embodiments, said first probe P_{A} is fully complementary to said mutant sequence of T_{M}.

In particular embodiments, the hybridisation sequence of said second probe P_{B} is selected using a theoretical model for ΔG calculation, preferably is designed and chosen based on a Nearest-Neighbor model, particularly comprising the steps of calculating or estimating the hybridisation free energy for the hybridisation between the target polynucleotide T_{wt} and probe P_{A}; followed by estimating or calculating the hybridisation free energy for the hybridisation between the target polynucleotide T_{wt} and a plurality of candidate probes P_{B} based on a Nearest-Neighbor model. Alternatively or additionally, probe P_{B} is selected via a hybridisation experiment between a plurality of candidate probes P_{B} and the target polynucleotide T_{wt}.

In particular embodiments, the methods as envisaged herein further comprise determining the relative amount of said target polynucleotide T_{wt} and said mutant polynucleotide T_{M} in said sample solution. Preferably, determining the relative amount of said target polynucleotide T_{wt} and said mutant polynucleotide T_{M} in said sample solution is performed using a calibration range for mutant polynucleotide T_{M}/target polynucleotide T_{wt} mixtures.

In particular embodiments said methods as envisaged herein further comprises determining which of a plurality of candidate mutant polynucleotides is present in said sample solution. Preferably, this comprises providing a plurality of probe pairs, wherein probe P_{A} of each probe pair is specific for a candidate mutant polynucleotide; obtaining a first and second measured hybridisation intensity I(P_{A})m and I(P_{B})m for each probe pair of said plurality of probe pairs; and comparing I(P_{A})m and I(P_{B})m for each probe pair of said plurality of probe pairs.

In particular embodiments, said sample solution as envisaged herein is prepared by:
extracting DNA from a sample of interest;
amplification of a target polynucleotide and mutant polynucleotide T_{M} thereof contained in said DNA using a pair of primers of which one primer has a phosphate modification at its 5′ end, thereby obtaining double stranded DNA; and
digesting the 5′ phosphate modified strands of said double stranded DNA using lambda exonuclease.

In particular embodiments, said hybridisation intensities of step (ii) are induced by emission of a label associated with a hybrid formed by binding of said target polynucleotide or mutants thereof and said probes. In particular embodiments, said label comprises a hybridisation sequence complementary to a sequence on said mutant polynucleotide T_{M} and said target polynucleotide outside said target sequence. In certain embodiments, said probe P_{A} and probe P_{B} as envisaged herein are differently labelled.

A second aspect of the present invention provides a computer program product for performing, when executed on a computing device, a method for determining the presence of a mutant T_{M} of a target polynucleotide in a sample solution according to the methods as envisaged herein, said computer program product being configured for
receiving hybridisation intensity I(P_{A})m for a sample solution with a first probe P_{A};
receiving hybridisation intensity I(P_{B})m for a sample solution with a second probe P_{B};
   analysing and comparing I(P_{A})m and I(P_{B})m, preferably determining the ratio I(P_{A})m/I(P_{B})m and comparing with the ratio I(P_{A})/I(P_{B}), and determining the presence of a mutant polynucleotide T_{M} in said sample solution based thereon.

Another aspect of the present invention relates to a device configured for performing the method for determining the presence of a mutant T_{M} of a target polynucleotide in a sample solution as envisaged herein, comprising one or more sets of reaction vessels, feeds for reagents connected thereto and a detection unit and a processing unit comprising the computer program product as envisaged herein.

The above and other characteristics, features and advantages of the concepts described herein will become apparent from the following detailed description, which illustrates, by way of example, the principles of the invention.

### BRIEF DESCRIPTION OF THE DRAWINGS

The following description of the figures of specific embodiments of the methods and instruments described herein is merely exemplary in nature and is not intended to limit the present teachings, their application or uses. Throughout the drawings, corresponding reference numerals indicate like or corresponding parts and features.

FIG. 1 shows a schematic representation of the concept of a particular embodiment of the present invention. A) schematic representation of the nucleotide sequences (target T_{wt} & mutant T_{M} sequences & probes P_{A} and P_{B}) used in the hybridisation; B) schematic representation of the hybridisation between probes and the nucleotide sequences in the sample, with the sample only containing the wild type, target polynucleotide; C) schematic representation of the hybridisation between probes and the nucleotide sequences in the sample, with the sample containing both the wild type, target polynucleotide and a mutant thereof.

FIG. 2 represents the ratio I(P_{A})/I(P_{B}) in function of the percentage of mutant nucleotide in a sample according to a particular embodiment of the present invention.

FIG. 3 represents the ratio I(P_{A})/I(P_{B}) in function of the percentage of mutant nucleotide in a sample according to a particular embodiment of the present invention.

### DETAILED DESCRIPTION OF THE INVENTION

While potentially serving as a guide for understanding, any reference signs used herein and in the claims shall not be construed as limiting the scope thereof.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” when referring to recited components, elements or method steps also include embodiments which “consist of” said recited components, elements or method steps.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments described herein are capable of operation in other sequences than described or illustrated herein.

The values as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to ensure one or more of the technical effects envisaged herein. It is to be understood that each value as used herein is itself also specifically, and preferably, disclosed. Typically, the term “about” should be read in this context, in particular in the context of the value of the ratio I(P_{A})/I(P_{B}).

The recitation of numerical ranges by endpoints includes all numbers and fractions subsumed within the respective ranges, as well as the recited endpoints.

All documents cited in the present specification are hereby incorporated by reference in their entirety.

Unless otherwise defined, all terms used in disclosing the concepts described herein, including technical and scientific terms, have the meaning as commonly understood by one of ordinary skill in the art. By means of further guidance, definitions for the terms used in the description are included to better appreciate the teaching of the present disclosure. The terms or definitions used herein are provided solely to aid in the understanding of the teachings provided herein.

The term “polynucleotide” as used herein may include oligonucleotides and refers to a polymer composed of nucleotide monomers, typically having a length of at least 10 nucleotides. Typically, the polynucleotides such as the target polynucleotides and probes referred to herein are single-stranded polynucleotides. As used herein, the term “polynucleotide” may include deoxyribonucleic acid (DNA), ribonucleic acid (RNA) or peptide nucleic acid (PNA).

The term “equilibrium” as used herein refers to thermodynamic equilibrium and indicates a situation wherein a steady state is obtained such that the number of conventional target-probe bindings does not substantially change over time. The term “non-equilibrium” or “non-equilibrium effects” refers to occurrence of a target-probe binding state that may change over time. The term “free energy” as used herein refers the Gibbs free energy (ΔG) or chemical potential.

The term “hybridisation” as used refers to nucleic acid hybridisation. This refers to the process of establishing a non-covalent sequence-specific interaction between two or more complementary strands of nucleic acids into a single hybrid. The strands of nucleic acids that may bind to their complement can for example be oligonucleotides, DNA, RNA or PNA. Nucleotides form the basic components of the strands of nucleic acids. Hybridisation comprises binding of two perfectly complementary strands (in the Watson-Crick base-pairing senses), but also binding of non-perfect complementary strands. With a non-perfect complementary strand reference may be made to strands having a small number of non-complementary elements such as one, two or more non-complementary elements, preferably one or two non-complementary elements. In principle there is no limit to the number of non-complementary elements but the more non-complementary elements, the easier these are detectable.

Reference throughout this specification to “one embodiment” or “an embodiment” means that a particular feature, structure or characteristic described in connection with the embodiment is included in at least one embodiment envisaged herein. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are also envisaged herein, and form different embodiments, as would be understood by those in the art. For example, in the appended claims, any of the features of the claimed embodiments can be used in any combination.

Provided herein are methods and systems for analyzing hybridisation for determining the presence of a mutant polynucleotide comprising a mutant sequence, also referred to herein as “mutant”, in a sample solution, particularly in a sample solution comprising both the mutant polynucleotide and the target, wild type polynucleotide. Accordingly, in a first aspect, the present application provides methods for determining the presence of a mutant polynucleotide in a sample solution preferably (also) comprising the wild type polynucleotide.

The term “mutant polynucleotide” or “mutant” (T_{M}) as used herein refers to a polynucleotide having a sequence (the mutant sequence) which differs from the sequence of a certain target ‘wild type’ polynucleotide (T_{wt}) in one or more nucleotides. It will be understood by the skilled person that the term “mutant” is not limited to sequences which are the result of a change in the target polynucleotide in a specific organism, tissue or cell but also include naturally occurring (i.e. evolutionary) sequence variants. More particularly in the context of the present application, these differences or mutations are located within a certain subsequence of the target polynucleotide, referred to herein as the “target sequence”. Again, it will be understood that the “target sequence” is the nucleotide sequence as present in the target wild type polynucleotide and is used as the reference sequence. In particular embodiments, the mutant polynucleotide T_{M} only differs from the target (wild type) polynucleotide T_{wt} in one or more nucleotides within the target sequence. Preferably, the mutant polynucleotide T_{M} differs from the target polynucleotide T_{wt} in a limited number of nucleotides within the target sequence, preferably in at most two nucleotides, such as in one or two nucleotides. In particular embodiments, the mutant polynucleotide differs from the target wildtype polynucleotide in only one nucleotide. Although the present method focuses on the interaction between a strand that initially is in a sample solution, and a strand that is bound to a surface, it is noted that hybridisation may occur between nucleic acid strands that both are in solution. The strands initially present in the sample solution and of interest for analysis are typically referred to in the art as the “target”, whereas the strand which is to hybridize to the target is referred to as “probe”. In the context of the methods provided herein, the mutant polynucleotide(s) T_{M} and target wild type polynucleotide T_{wt} referred to herein are both potentially present in the sample and may both be considered as “targets”. In preferred embodiments both the mutant polynucleotide(s) T_{M} and target wild type polynucleotide T_{wt} referred to herein are both present in the sample. The probe may for example be an oligonucleotide, such a DNA, RNA or PNA sequence (partially) complementary to a target. In the methods envisaged herein, the probes may be contacted with the target in the sample by introducing the probes into the sample solution. Preferably, the probe is preferably bound to a surface, such as a carrier, particularly on a microarray.

However, in particular embodiments of the present methods the probes are present in solution. Typically, the probes as envisaged for use herein are different (single-stranded) probes having different binding affinities for the (target sequence of the) target polynucleotide T_{wt} and the mutant polynucleotide Tₘ. It will be understood by the skilled person however that the reference to the “target” polynucleotide and the “mutant” polynucleotide is arbitrary in that they can be interchanged.

The probes envisaged for use in the methods provided herein may contain a hybridisation sequence. The hybridisation sequence is the sequence intended for hybridisation with the target sequence and thus its sequence will be determined by the target sequence. Optionally, the probe may further comprise a tail sequence, which may be used to hybridize to other sequences, for tagging of the probe, etc. . . . . The length of the hybridisation sequence typically corresponds to the target sequence, i.e. contains the same number of nucleotides. A so-called “perfect match probe” as used herein refers to a probe having a hybridisation sequence which is completely complementary to a target sequence of a target polynucleotide. The term “mismatch probe” as used herein refers to a probe having a hybridisation sequence which is non-complementary to the target sequence. The probe is considered non-complementary as soon as one nucleotide differs from the target sequence of the target polynucleotide. In particular embodiments, the hybridisation sequence of the probe comprises one or more, such as at most two, non-complementary nucleotides with respect to the target sequence in the target polynucleotide.

The methods for determining the presence of a mutant polynucleotide T_{M} in a sample solution as envisaged herein comprise the steps of contacting the sample with two different probes which differ in their complementarity to the target and mutant sequence. The present inventors have found that by comparing the hybridisation intensities of hybridisation experiments on a sample solution using a specifically designed or selected probe pair, it is possible to identify mutant polynucleotides in a mixture of mutant T_{M} and wild type polynucleotide T_{wt} at low concentrations of the mutant relative to the wild type, with a minimal set of probes without the need of a parallel reference measurement or a reference sample. The methods are based on the observation that the detection of a mutant sequence in a sample can be detected easily and without the need for a reference sequence provided that two probes are used for which the ratio of the hybridisation intensity for the hybridisation with the target is known. Indeed, where the ratio of hybridisation intensities of the different probes is known, it is possible to easily identify the presence or absence of a mutant in the sample.

Accordingly, the methods of the present invention are based on the provision of a suitable pair of probes. More particularly the methods encompass the step of designing and/or selecting probes, named probes P_{A} and P_{B} herein. The probe pair P_{A} and P_{B} can be designed such that each probe (and more particularly the hybridisation sequence of the probe) contains a (different) level of mismatch with the wild type target sequence. Accordingly, more particularly, the methods provided herein comprise the steps of:

(i) providing a probe pair consisting of a first probe P_{A} and a second (different) probe P_{B};

(ii) contacting a sample solution with said first probe P_{A} and said second probe P_{B}, and obtaining or detecting hybridisation intensities I(P_{A})m and I(P_{B})m for said first probe P_{A} and said second probe P_{B} of said probe pair, respectively; and

(iii) analysing and comparing I(P_{A})m and I(P_{B})m and determining the presence of said mutant polynucleotide T_{M} in said sample solution based thereon.

More particularly, in these methods a first probe P_{A} and a second (different) probe P_{B} are designed or selected such that the hybridisation sequences of said first probe P_{A} and second probe P_{B} are characterized in that

(i) they each comprise at least one non-complementary nucleotide with respect to the target sequence. In particular, the at least one non-complementary nucleotide of probe P_{A} (vs the target sequence of T_{wt}) is different from the non-complementary nucleotide of probe P_{B} (vs the target sequence of T_{wt});
the hybridisation sequence of said first probe P_{A} comprises one or more nucleotides complementary to the one or more nucleotides of the mutant sequence differing from said target sequence; and that the ratio of the hybridisation intensity [I(P_{A})] for the hybridisation between the target polynucleotide and the first probe P_{A} to the hybridisation intensity [I(P_{B})] for the hybridisation between the target polynucleotide and the second probe P_{B} is a known value. In preferred embodiments, said first probe P_{A} and second probe P_{B} are selected so that the ratio of the hybridisation intensity [I(P_{A})] for the hybridisation between the target polynucleotide and the first probe P_{A} to the hybridisation intensity [I(P_{B})] for the hybridisation between the target polynucleotide and the second probe P_{B} ranges between 0.02 and 50, more preferably between 0.05 and 20 or more particularly between 0.1 and 10;

In particular embodiments, said first probe P_{A} and second probe P_{B} are selected such that only said first probe P_{A} has a hybridisation sequence specific for the mutant sequence, i.e. comprising at least one nucleotide complementary to the at least one nucleotide of the mutant sequence differing from the target sequence of the target wild type polynucleotide T_{wt}; and that the ratio of the hybridisation intensity [I(P_{A})] for the hybridisation between the target polynucleotide T_{wt} and the first probe P_{A} to the hybridisation intensity [I(P_{B})] for the hybridisation between the target polynucleotide T_{wt} and the second probe P_{B} is a known value known and ranges between 0.02 and 50, more preferably between 0.05 and 20 or between 0.1 and 10. Preferably, the first probe P_{A} and second probe P_{B} have a hybridisation sequence with each at least one different non-complementary nucleotide with respect to the target, wild type sequence T_{wt}.

In particular embodiments, probe P_{A} comprises a nucleotide complementary to the one or more nucleotides differing between the sequence of the wild type and the mutant polynucleotides, or stated differently, probe P_{A} is specific for the mutant polynucleotide, whereas this is not the case for probe P_{B}, which comprises a non-matching nucleotide to the mutant nucleotide. In preferred embodiments, said first probe P_{A} is fully complementary to the mutant sequence of the mutant polynucleotide. As used herein, probe P_{A} is also referred to as the “mutant probe”.

In addition, the hybridisation sequence of probe P_{B} comprises a suitable sequence variation of the target (wild type) sequence T_{wt} such that the ratio I(T_{wt},P_{A})/I(T_{wt},P_{B}) is a known value, preferably ranging between 0.02 and 50, more preferably between 0.05 and or most particularly between 0.1 and 10, even more preferably ranging between 0.5 and 2. In particularly preferred embodiments, Probe P_{B} is selected or designed such that I(T_{wt},P_{A}) is about equal I(T_{wt},P_{B}), or stated differently wherein the ratio I(T_{wt},P_{A})/I(T_{wt},P_{B}) is about 1 (or 1+/−50%, such as 1+/−20% or 1+/−10%).

In certain embodiments, probe P_{B}, and optionally also probe P_{A}, is designed using a theoretical model for ΔG calculation, preferably using a Nearest Neighbor model ((Bloomfield Va. et al., “Nucleic Acids Structures, Properties and Functions, University Science Books, Mill Valley, 2000). This is possible since a stringent relation (e.g. the Langmuir isotherm under certain experimental conditions) exists between measured intensities I and the corresponding free energy ΔG. Nearest neighbor models provide a good estimation of the hybridisation free energy as a sum of dinucleotide parameters. In these embodiments, the method may comprise calculating or estimating the hybridisation free energy for the hybridisation between the wild type target polynucleotide T_{wt} and probe P_{A}; followed by estimating or calculating the hybridisation free energy for the hybridisation between the wild type target polynucleotide T_{wt} and a plurality of candidate probes P_{B} based on a Nearest-Neighbor model. In particular embodiments, probe P_{B} is selected following this theoretical calculation as the probe for which ΔG(T_{wt},P_{B}) minus ΔG(T_{wt},P_{A}) is minimal, more preferably as the probe for which ΔG(T_{wt},P_{B}) is about equal to ΔG(T_{wt},P_{A}) (or wherein ΔG(T_{wt},P_{B}) minus ΔG(T_{wt},P_{A}) is about 0). It is an advantage of embodiments according to the present invention that hybridisation free energy can be determined accurately for hybridisation between a target initially in solution and a probe bound to a surface, such as for example may occur in microarrays. Advantageously, using a theoretical model is a quick and straightforward way to design probe P_{B}. It will be understood that in what follows when referring to the nature of the probes, more particularly to their ability to hybridize with a target sequence, this in fact refers to the hybridisation sequence of the probes.

In certain embodiments, probe P_{B} is selected via an experiment which allows the evaluation of ΔG(T_{wt},P_{A}) and ΔG(T_{wt},P_{B}), and hence allows the evaluation of the ratio ΔG(T_{wt},P_{A})/AG(T_{wt},P_{B}), and accordingly the ratio I(T_{wt},P_{A})/I(T_{wt},P_{B}). For instance this can be done by a hybridisation experiment between different (individual) probes and the target wildtype polynucleotide T_{wt}. Advantageously, said experiment is a microarray experiment, allowing many sequences to be evaluated in parallel. For instance the array can be carried out with a test sample comprising only target wildtype polynucleotide (T_{wt}) and whereby one spot of the array contains probe P_{A} and wherein other spots of the array contain each individually a different candidate probe P_{B}. For instance, the different candidate probes P_{B} can be complementary to the wild type except for at least one nucleotide variation. Preferably, probe P_{B} is selected based on the fact that it corresponds to the candidate probe P_{B} which gives a hybridisation intensity with the target nucleotide T_{wt} comparable to or about equal to the hybridisation intensity of probe P_{A} with the target polynucleotide T_{wt}. In certain embodiments, probe B is selected via theoretical modelling and calculation combined with experimental data.

In certain embodiments, in case additional degrees of freedom in the selection of suitable probes P_{A} and P_{B} are required, probe P_{A} can be designed to be specific for the mutant polynucleotide T_{wt}, i.e. comprising a nucleotide complementary to the one or more nucleotides differing between the sequence of the wild type and the mutant polynucleotides, but comprises in addition one or more nucleotides non-complementary to the target wild type polynucleotide T_{wt}. Probe P_{B} is designed or selected as described above.

The methods as envisaged herein are of particular interest for the analysis of a sample solution which comprises a mixture of the target polynucleotide [T_{wt} or wild type (wt)] and a mutant polynucleotide [T_{M} or (mut)]. More particular they are of interest in samples which are characterized in that the concentration of the mutant polynucleotide [c(mut)] is significantly smaller than the concentration of the target or wild type polynucleotide [c(wt)]. The ratio of these concentrations [c(mut)/c(wt)] is also referred to herein as the “relative concentration” of mutant polynucleotide in the sample solution. In preferred embodiments, the relative concentration of mutant polynucleotide in the sample solution is between 0.01 and 0.5, more preferably between 0.01 and 0.1. The relative concentration may also be expressed as a percentage, which refers to 100*[c(mut)/c(wt)].

The sample solution may be prepared for use in the methods envisaged herein using standard methods known in the art. This may include extracting DNA or other polynucleotides from a sample of interest, followed by amplification of certain fragments within the extracted DNA. Typically, amplification is performed using PCR (polymerase chain reaction). However, this results in double stranded DNA, whereas single-stranded DNA is preferred for the present methods. Indeed, hybridisation of double-stranded DNA with nucleic acid probes is hampered by competition between the complementary non-target strand and the probe. Such competition can be avoided by degradation of the complementary strands, for example using lambda exonuclease. Lambda exonuclease is a processive enzyme that acts in the 5′ to 3′ direction, catalyzing the removal of 5′ mononucleotides from duplex DNA. The preferred substrate is 5′-phosphorylated double stranded DNA. Accordingly, in certain embodiments, the preparation of the sample solution may comprise the steps of:
extracting DNA from a sample of interest;
amplification of a target polynucleotide and mutants thereof contained in said DNA using a pair of primers of which one primer has a phosphate modification at its 5′ end; thereby obtaining double stranded DNA; and
digesting the 5′ phosphate modified strands of said double stranded DNA using lambda exonuclease.

The contacting of the sample with the probes (referred to as step (ii) above) is typically performed under conditions suitable for hybridisation of the target and mutant polynucleotides to said probes. The skilled person understands that relevant parameters for optimizing hybridisation include hybridisation time, temperature, and probe length. In preferred embodiments, the probes have a length ranging from about 20 to about 30 nucleotides. Furthermore, it is preferred that the hybridisation experiments are performed under such conditions that hybridisation has reached equilibrium, e.g. by selecting suitable probe lengths, temperatures, and hybridisation time. A method for determining for which probes or spots the hybridisation has reached equilibrium is described in international patent application WO2011/035801, which is hereby incorporated by reference in its entirety.

Although the methods envisaged herein may be carried out by using probes which are added to the sample in solution, it is preferred that the probes are provided on a surface. The probes may be provided on any type of carrier, such as magnetic beads or fibers. However, it is preferred that the probes are provided on a microarray. Thus, in particular embodiments of the methods described herein, the first and second probe of the probe pair are provided on separate spots of a microarray. A microarray as a hybridisation platform contains a large number of probes which are immobilized on a solid surface. Preferably, the probes are provided in spatially separated spots, wherein each spot comprises one (and only one) type of probe. Typically, each spot comprises only a few picomoles of each probe. Typical microarrays comprise hundreds or even thousands of spots. A plurality of microarray platforms suitable for use in the present methods are commercially available, and include but are not limited to the platform provided by Agilent, the GeneChips platform from Affymetrix or CodeLink Bioarray platform from Amersham Biosciences.

In preferred embodiments, the first and second probes may be provided on the same microarray. This can facilitate comparing the hybridisation intensities for the probe pair.

The hybridisation intensity as envisaged herein is a value representing the fraction of a certain probe which is hybridized. In the methods described herein, detection of hybridisation intensity may be performed using a marker associated with the formed hybrid, such as for example a fluorescence marker or a radio-active marker, or other markers known in the art. In preferred embodiments, the marker used for probe P_{A} is the same as the marker used for probe P_{B}. However, this is not critical for the present methods. Accordingly, different markers may be used for probe P_{A} and probe P_{B} of the probe pair. In certain embodiments, the detection of the hybridisation intensity may be performed using a label-free method as known by the skilled person, such as surface-enhanced Raman spectroscopy.

Typically in hybridisation experiments, intensity of the radiation or fluorescence provided by the markers is detected and representative for the number of hybrids formed. Thus, in certain embodiments, the hybridisation intensities may be induced by emission of a label associated with a hybrid formed by binding of the target polynucleotide or mutant thereof and said probes. Suitable fluorescence markers for the present methods include, but are not limited to, Cy3 and Cy5, which are dyes of the cyanine dye family.

The markers or labels may be associated to the target or mutant polynucleotide prior to or after hybridisation, such as during PCR amplification of the sample DNA. In some embodiments, a fluorescent dye or other marker compound may be associated directly to the target or mutant thereof. In other embodiments, the marker compounds may be associated to the target or mutant thereof in an indirect manner, for example via a “barcode”, which is a strand having a hybridisation sequence which is complementary to a tail sequence which is present on the mutant polynucleotide of interest and on the target polynucleotide, thereby allowing hybridisation between the barcode and target (or mutant thereof), and therefore indirect coupling of the fluorescence marker or other marker to the target. More particularly, the strand hybridizes to a tail sequence outside the target sequence of the target polynucleotide, such that it does not significantly interfere with the hybridisation between the targets and the probes.

After the detection of the signal resulting from the hybridisation of the sample DNA with the probes, the analysis is performed. In particular embodiments, the analysis (referred to as step (iii) above) of the present methods, comprises the comparison of the measured hybridisation intensities of the sample solution with each probe of the probe pair. Thus, the measured intensity of the hybridisation of the sample solution with probe P_{A} [I(P_{A})m] is compared to the measured intensity of the hybridisation of the sample solution with probe P_{B} [I(P_{B})m]. Based on the comparison of said hybridisation intensities, particularly by determining the ratio of the measured intensity of the hybridisation with probe P_{A} vs the measured intensity of the hybridisation with probe P_{B} [i.e. I(P_{A})m/I(P_{B})m], the presence of one or more mutant polynucleotides can be determined.

In particular embodiments, the hybridisation intensities may be analyzed using statistical methods, wherein I(P_{A})m/I(P_{B})m is compared to the known ratio I(P_{A})/I(P_{B}) of step (i).

Indeed, in case I(P_{A})m/I(P_{B})m deviates from, particularly is higher than, the ratio I(P_{A})/I(P_{B}) of step (i), this corresponds to the presence of a mutant polynucleotide in the sample solution. A deviation from the ratio I(P_{A})/I(P_{B}) of step (i) may be a deviation, preferably increase, of at least 5%, more preferably of at least 10%, still more preferably of at least 25%, even more preferably of at least 33%.

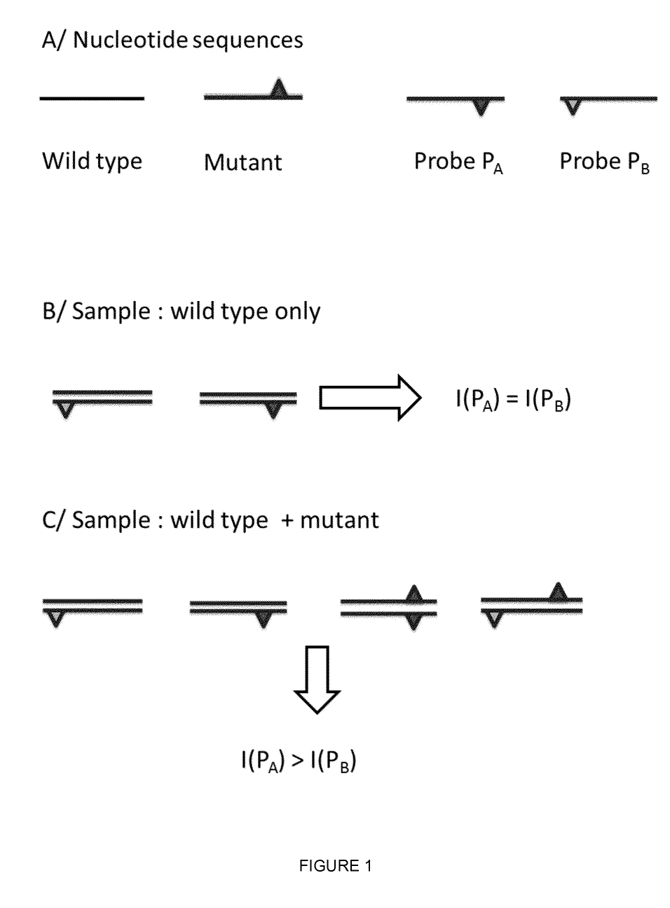

In this context, specific ways to detect and/or identify mutant polynucleotides in a sample solution will be explained below, using theoretical concepts based on the thermodynamics of DNA hybridisation, as illustrated for a particular embodiment in FIG. 1. The skilled person will understand that this also applies, mutatis mutandis, to nucleotides other than DNA. While in the following, the theoretical concept is explained in the context of microarray data analysis, the skilled person will understand this also applies for other experimental setups for measuring/analysing hybridisation, including hybridisation in solution.

In microarray data analysis, each probe intensity (hybridisation intensity) is associated to a signal from a spot. A spot is a local space on the microarray slide that contains a large number of identical sequences corresponding to a certain type of probe in the probe set. Therefore, typically, each spot represents a single type of probe. Each of these identical sequences within a spot is supposed to be hybridized to a floating target sequence depending on the affinity between the two sequences. This affinity is sequence dependent and determines the fraction of hybridized probes in a spot.

In a hybridisation model with Langmuir isotherm (Hooyberghs et al., Nucleic Acids Res. 2009, 37, e53), the relationship between the detected intensity and the hybridisation affinity for the target-probe hybrid can approximately be written as equation (1) (assuming the hybridisation between the target and probe sequences are in thermodynamic equilibrium, and that the fraction of hybridized probes in a microarray spot is such that the detected intensities are significantly above the background yet far from saturation):

I=A.e^{-ΔG/RT} (1)

wherein I is the detected hybridisation intensity, A is a proportionality factor for the intensity and is e.g. system dependent, ΔG is hybridisation free energy as a sequence dependent measure for the affinity, i.e. the free energy difference between two ssDNA sequences and their DNA duplex formed by the hybridisation, R is the ideal gas constant, and T is the experimental temperature.

This equation can be calculated for each spot of the microarray, i.e. for each probe type in a probe set.

In case of the hybridisation between a sample containing either a target (wild type) sequence (T_{wt}) or either a mixture of a target sequence T_{wt} and a mutant sequence Tₘ and a probe pair, P_{A} and P_{B}, wherein probe P_{A} comprises a sequence which is fully complementary to the mutant sequence Tₘ and wherein probe P_{B} comprises a sequence which is fully complementary to the wild type target sequence T_{wt} except for at least one (different) variation, the following relations can be deduced.

When a wild type target sequence T_{wt} hybridizes to probe P_{A}, the Langmuir isotherm (equation 1) representing the intensity of the spot corresponding to this probe can be described as follows:

I=A.e^{-ΔG(T}^{_{WT,}}^{P}^{_{A}}^{)/RT} (2)

With −ΔG(T_{wt},P_{A}) a measure of the affinity between probe P_{A} and the wild type target T_{wt}. As the P_{A}−T_{wt} duplex contains at least one mismatch (P_{A} is fully complementary to a mutant sequence Tₘ, differing in at least one nucleotide from the wild type target T_{wt}), which gives an affinity penalty and a corresponding reduction of the hybridisation intensity (cf. Langmuir isotherm; equation 1 and 2)

A similar situation occurs for the hybridisation between the wild type target sequence T_{wt} and probe P_{B}, and the corresponding Langmuir isotherm (equation 1) can be described as follows:

I=A.e^{-ΔG(T}^{_{WT}}^{,P}^{_{B}}^{)/RT} (3)

With −ΔG(T_{wt},P_{B}) a measure of the affinity between probe P_{A} and the wild type target T_{wt}.

Equations (2) and (3) allow to select and/or design a probe P_{B} (e.g. by including at least one well-chosen mismatch different from a mutant nucleotide) so that in a particular embodiment ΔG(T_{wt},P_{A})=ΔG(T_{wt},P_{B}), via theoretical modelling (e.g. via the nearest neighbor model) or by performing hybridisation experiments, or stated differently, wherein the ratio ΔG(T_{wt},P_{A})/AG(T_{wt},P_{B})=1.

(1) Thus, for a sample containing only wild type target sequence T_{wt}, since ΔG(T_{wt},P_{A})=ΔG(T_{wt},P_{B}), the affinity of probes P_{A} and P_{B} with respect to the target sequence T_{wt} are equal (FIG. 1B). Hence, as follows from equations (2) and (3), the measured intensities after a hybridisation experiment with only wild type target T_{wt} in the sample will be equal: I(P_{A})=I(P_{B}), or, stated differently, the ratio I(P_{A})/I(P_{B})=1.

(2) In the case of a hybridisation where the sample contains mixture of a target and mutant sequences i.e. a wild type sequence and a mutant such as in a clinical situation, there will be a competition between the two target sequences to hybridize to a single probe. To describe this competitive hybridisation, equation 1 can be extended to:

I=I(wt)+I(mut)=A.e^{-ΔG(wt)/RT}+A.e^{-ΔG(mut)/RT} (4)

wherein I(wt) is the wild type contribution to the total signal and I(mut) is the mutant contribution, ΔG(wt) is free energy of the wild type and ΔG(mut) is free energy of the mutant.

Accordinlgy, if a sample contains the wild type target sequence T_{wt} and a mutant sequence Tₘ, both sequences will hybridize to each probe, and the resulting hybridisation intensity for probe P_{A} and probe P_{B} can be described as in equation 5 and 6, respectively:

I(P_{A})=A.[e^{-ΔG(T}^{_{WT}}^{,P}^{_{A}}^{)/RT}+e^{-ΔG(T}^{_{M}}^{,P}^{_{A}}^{)/RT]} (5)

I(P_{B})=A.[e^{-ΔG(T}^{_{WT}}^{,P}^{_{B}}^{)/RT}+e^{-ΔG(T}^{_{M}}^{,P}^{_{B}}^{)/RT]} (6)

As before, the contribution of the first term (relating to the hybridisation between the wild type target T_{wt} to each probe) is equal for both probe P_{A} and P_{B}, but the contribution of the second term to the hybridisation intensity is highly different for the different probes:

With the sequence of probe P_{A} fully matching the mutant sequence T_{wt}, they will have a much higher affinity to bind: i.e. P_{A}-Tₘ affinity is high. In contrast, the sequence of probe P_{B} contains at least two mismatches with respect to T_{M} (i.e. the at least one nucleotide differing between T_{wt} and Tₘ, and an additional mismatch nucleotide), and consequently the P_{B}-Tₘ affinity is low (FIG. 10).

Accordingly, ΔG(Tₘ,P_{A})>ΔG(Tₘ,P_{B}), and consequently, I(P_{A})>I(P_{B}), or, stated differently, the ratio I(P_{A})/I(P_{B})>1.

This reasoning clearly shows that even when limited amounts of the mutant sequence are present, the hybridisation intensities of the two probes with the sample solution will no longer be equal. Advantageously, by calculating the ratio of the intensities with probe P_{A} vs probe P_{B}, the analysis is independent from the proportionality factor A, and thus there is no need to analyse a reference sample.

While in this setup the theoretical concept is explained in terms of I(P_{A})/I(P_{B})=1, the skilled person will understand this also applies for other setups where the ratio I(P_{A})/I(P_{B}) is known, but not equal to 1: in this case, even at low levels of the mutant sequence in the sample solution, the ratio I(P_{A})/I(P_{B}) will increase as well and the measured ratio I(P_{A})m/I(P_{B})m will deviate from the known ratio I(P_{A})/I(P_{B}).

In particular embodiments of the present invention, the method further comprises determining the relative amount of said target polynucleotide and said mutant polynucleotide in said sample solution, for instance by using a calibration range for mutant polynucleotide/target wild type polynucleotide mixtures.

In certain embodiments, the present methods may comprise determining which of a plurality of candidate mutant polynucleotides is present in the sample solution. In this context, the method comprises contacting the sample solution with a plurality of probe pairs as envisaged herein, wherein each probe pair comprises a probe P_{A} specific for one of the candidate mutant polynucleotides and a probe P_{B} designed or selected as above. It is understood that the plurality of probe pairs may comprise a same or different probe P_{B}. Next, the mutant polynucleotide(s) present in said sample can be identified and determined based on the probe pair(s) out of said plurality of probe pair(s) for which the ratio I(P_{A})m/I(P_{B})m deviates from the ratio I(P_{A})/I(P_{B}) of step (i)

Further provided herein is a computer program product for performing, when executed on a computing device, at least a part of a method for determining the presence of a mutant of a target polynucleotide as described herein. For example, the computer programs may be configured for receiving and analyzing hybridisation intensities according to the methods described herein. For example, the computer program may be configured to analyse the intensity of the hybridisation of the sample solution with a first and second probe of a probe pair, and to statistically analyse and compare the ratio of the hybridisation intensities with the first and second probe as described herein. In particular embodiments, the computer program product is configured for receiving a hybridisation intensity for a sample solution with a first probe P_{A} of a probe pair, for receiving a hybridisation intensity for a sample solution with a second probe P_{B} of said probe pair and analyzing the ratio of the measured intensities and comparing it to the (theoretical) ratio of the hybridisation intensities of the wild type polynucleotide T_{wt} with probes P_{A} and P_{B}, calculated using e.g. a Nearest Neighbor model.

The software may further be configured to perform a statistical analysis of the hybridisation intensity data using a first and second probe of one or more probe pairs in order to determine which of a plurality of candidate mutants is present in a sample solution. In certain embodiments, the computer programs may further be configured for designing suitable probe sets based on information of the target sequence and/or mutations thereof, particularly based on a Nearest Neighbor model for determining the difference in free energy.

In case of implementation or partly implementation as software, such software may be adapted to run on suitable computer or computer platform, based on one or more processors. The software may be adapted for use with any suitable operating system. The computing means may comprise a processing means or processor for processing data.

A further tool provided herein is a device configured for carrying out the methods provided herein. More particularly the device comprises the combination of the necessary hardware and software for carrying out the different steps of these methods. The device may comprise hardware, in the form of reaction vessels and feeds for reagents connected thereto and a detection unit, which can ensure the contacting a sample solution with a first probe P_{A} and a second probe P_{B}, and hybridisation of the sample solution with said probe P_{A} and said probe P_{B}. Moreover, the device comprises a processing unit provided with the necessary software for performing the analysis step involving the comparison of the intensity measurements with the first and second probe of a probe pair and optionally a display unit to present the results of said analysis to a user. In particular embodiments, the results are displayed as information on the presence of a mutant polynucleotide in the sample solution.

This concept is further exemplified in the examples below.

### Examples

The following examples are provided for the purpose of illustrating the claimed methods and applications and by no means are meant and in no way should be interpreted to limit the scope of the present invention.

The inventors have applied the present method for the detection of point mutations in the K-RAS oncogene, which is an important genetic marker for colorectal and lung cancer diagnostics and treatment stratification.

The nucleotide sequence of the target polynucleotide (wild type K-RAS SEQ ID No 1) and a mutant polynucleotide (SEQ ID No 2) are as follows, with the target and mutant sequence being underlined and the nucleotide in the mutant sequence differing from the wild type target sequence marked by (*) (“mutant polynucleotide”):

Wild type (SEQ ID No 1):
GTCCTGCACCAGTAATATGCATATTAAAACAAGATTTACCTCTATTGTTG
GATCATATTCGTCCACAAAATGATTCTGAATTAGCTGTATCGTCAAGGCA
CTCTTGCCTACGCCACCAGCTCCAACTACCACAAGTTTATATTCAGTCAT
TTTCAGCAGGCCTTATAATAAAAATAATGAAAATGTGACTATATTAGAAC
ATGTCACACATAAGGTTAATAC
Mutant (SEQ ID No 2)
GTCCTGCACCAGTAATATGCATATTAAAACAAGATTTACCTCTATTGTTG
GATCATATTCGTCCACAAAATGATTCTGAATTAGCTGTATCGTCAAGGCA
CTCTTGCCTACGCCAG(*)CAGCTCCAACTACCACAAGTTTATATTCAGT
CATTTTCAGCAGGCCTTATAATAAAAATAATGAAAATGTGACTATATTAG
AACATGTCACACATAAGGTTAATAC

In a first exemplary setup, a probe pair was designed with probe P_{A} being a perfect match probe for the mutant sequence and probe P_{B} being fully complementary to the target sequence, except for one nucleotide, different from the mutant nucleotide (nucleotides not complementary to the target sequence are underlined):

(SEQ ID No3)
Perfect match probe: P_{T}: GTTGGAGCTGGTGGCGTAGGCAA
(SEQ ID No4)
Mutant match probe: P_{A}: GTTGGAGCTGCTGGCGTAGGCAA
(SEQ ID No5)
“Insensitive” probe: P_{B}: GTTGGGGCTGGTGGCGAAGGCAA

A hybridisation experiment with a sample comprising only the wild type polynucleotide and 0% mutant polynucleotide was performed and the ratio of the hybridisation intensity of the hybridisation of probe P_{A} with said sample (I_{A}) to the ratio of the hybridisation intensity of the hybridisation of probe P_{B} with said sample (I_{B}) was measured: the ratio I_{A}/I_{B} equaled 1.0229. This shows that with a one-point mutation a probe P_{B} can be obtained having about the same hybridisation intensity for hybridisation with the target (wild type) sequence as probe P_{A}.

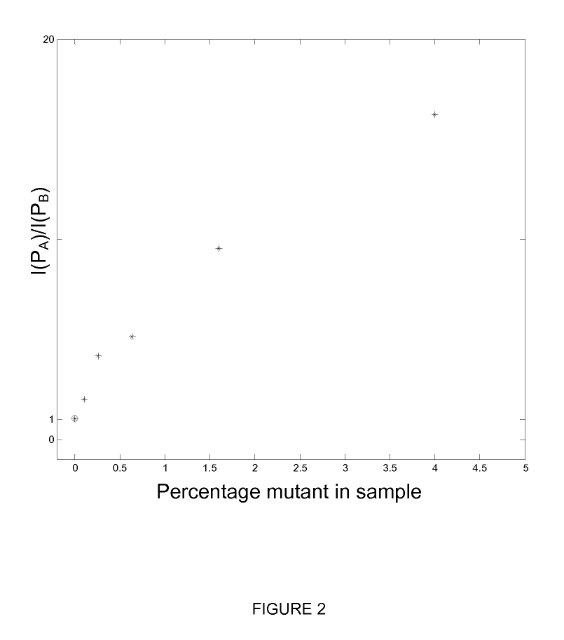

When performing hybridisation experiments with samples with increasing levels of mutant polynucleotide, I_{A} increases, due to the perfect match hybridisation between probe P_{A} and the mutant polynucleotide, and I_{B} decreases due to the mutant sequence presenting two non-complementary nucleotides with probe P_{B}. Accordingly, the ratio I_{A}/I_{B} increases: I_{A}/I_{B}>1. This is represented in FIG. 2.

In a second exemplary setup, a probe pair was designed with probe P_{A} being fully complementary to the mutant target sequence except for one nucleotide and probe P_{B} being fully complementary to the target sequence, except for two nucleotides, different from the non-matching nucleotides of probe P_{A}: (nucleotides not complementary to the target sequence are underlined):

(SEQ ID No3)

| | |
|---|---|
| Perfect match probe: | P_{T}: GTTGGAGCTGGTGGCGTAGGCAA |

(SEQ ID No6)

| | |
|---|---|
| Mutant probe: | P_{A}: GTTGGAGCTGCTGGCGGAGGCAA |

(SEQ ID No7)

| | |
|---|---|
| “Insensitive” probe: | P_{B}: GTTGGAGTTGGTGGCGTCGGCAA |

A hybridisation experiment with a sample comprising only the wild type polynucleotide and 0% mutant polynucleotide was performed and the ratio of the hybridisation intensity of the hybridisation of probe P_{A} with said sample to the ratio of the hybridisation intensity of the hybridisation of probe P_{B} with said sample was measured and yielded a ratio I_{A}/I_{B}=1.0126. This shows that a probe pair can be obtained having a very similar hybridisation intensity for hybridisation with the target (wild type) sequence.

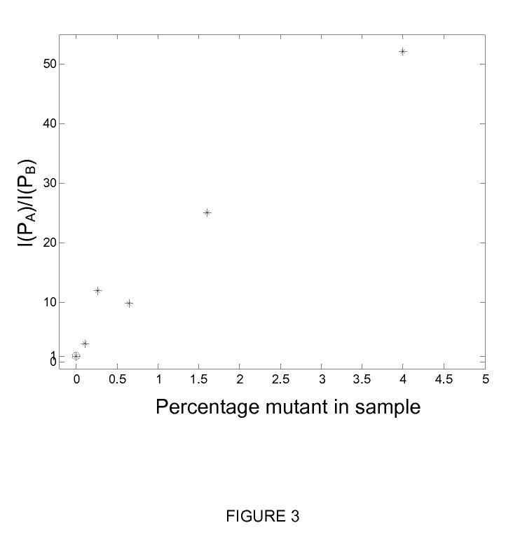

When performing hybridisation experiments with samples with increasing levels of mutant polynucleotide, I_{A} increases, and I_{B} decreases, as above. Accordingly, the ratio I_{A}/I_{B} increases with increasing mutant polynucleotide concentration. This is represented in FIG. 3.

1. A method for determining the presence of a mutant polynucleotide T_{M} in a sample solution, said mutant polynucleotide T_{M} comprising a mutant sequence and differing from a target polynucleotide T_{wt} comprising a target sequence in one or more nucleotides of said target sequence, said method comprising:
providing a probe pair comprising a first probe P_{A} and a second different probe P_{B}, wherein
the hybridisation sequences of said first probe P_{A} and second probe P_{B} are characterized in that the first probe P_{A} comprises at least one non-complementary nucleotide (NT1) with respect to the target sequence of T_{wt} and the second probe P_{B} comprises at least one different non-complementary nucleotide (NT2) with respect to the target sequence of T_{wt};
the hybridisation sequence of said first probe P_{A} comprising one or more nucleotides complementary to the one or more nucleotides of the mutant sequence of T_{M} differing from said target sequence of T_{wt}; and
wherein the ratio of the hybridisation intensity [I(P_{A})] for the hybridisation between the target polynucleotide T_{M} and the first probe P_{A} to the hybridisation intensity [I(P_{B})] for the hybridisation between the target polynucleotide T_{M} and the second probe P_{B} is a known value, wherein at least the probe P_{B} is selected using a theoretical model for ΔG calculation, preferably using a Nearest Neighbor Model, and/or wherein probe P_{B} is selected via a hybridisation experiment between a plurality of candidate probes P_{B} and the target polynucleotide T_{wt};
(ii) contacting said sample solution with said first probe P_{A} and second probe P_{B} of said probe pair, and obtaining a first and second measured hybridisation intensity I(P_{A})m and I(P_{B})m for said first and second probe, respectively; and
(iii) analysing and comparing I(P_{A})m and I(P_{B})m and determining the presence of said mutant polynucleotide T_{M} in said sample solution based thereon.

2. The method according to claim 1 wherein said first probe P_{A} and second probe P_{B} are selected so that the ratio of the hybridisation intensity [I(P_{A})] for the hybridisation between the target polynucleotide T_{wt} and the first probe P_{A} to the hybridisation intensity [I(P_{B})] for the hybridisation between the target polynucleotide T_{wt} and the second probe P_{B} is known and ranges between 0.02 and 50.

3. The method according to claim 1, wherein said first probe P_{A} is fully complementary to said mutant sequence T_{M}.

4. The method according to claim 1, wherein the hybridisation sequence of said second probe P_{B} is chosen based on a Nearest-Neighbor model comprising the steps of calculating or estimating the hybridisation free energy for the hybridisation between the target polynucleotide T_{wt} and probe P_{A}; followed by estimating or calculating the hybridisation free energy for the hybridisation between the target polynucleotide T_{wt} and a plurality of candidate probes P_{B} based on a Nearest-Neighbor model.

5. The method according to claim 1, wherein the ratio I(P_{A})/I(P_{B}) is about 1.

6. The method according to claim 1, further comprising determining the relative amount of said target polynucleotide T_{wt} and said mutant polynucleotide T_{M} in said sample solution.

7. The method according to claim 6 wherein determining the relative amount of said target polynucleotide T_{wt} and said mutant polynucleotide T_{M} in said sample solution is performed using a calibration range for mutant polynucleotide T_{M}/target polynucleotide T_{wt} mixtures.

8. The method according claim 1 further comprising determining which of a plurality of candidate mutant polynucleotides is present in said sample solution.

9. The method according to claim 8, comprising providing a plurality of probe pairs, wherein probe P_{A} of each probe pair is specific for a candidate mutant polynucleotide; obtaining a first and second measured hybridisation intensity I(P_{A})m and I(P_{B})m for each probe pair of said plurality of probe pairs; and comparing I(P_{A})m and I(P_{B})m for each probe pair of said plurality of probe pairs.

10. The method according to claim 1, wherein said sample solution is prepared by:
extracting DNA from a sample of interest;
amplifying a target polynucleotide T_{wt} and mutant polynucleotide T_{M} thereof contained in said DNA using a pair of primers of which one primer has a phosphate modification at its 5′ end, thereby obtaining double stranded DNA; and
digesting the 5′ phosphate modified strands of said double stranded DNA using lambda exonuclease.

11. The method according to claim 1, wherein said hybridisation intensities in step (ii) are induced by emission of a label associated with a hybrid formed by binding of said target polynucleotide or mutants thereof and said probes.

12. The method according to claim 11, wherein said label comprises a hybridisation sequence complementary to a sequence on said mutant polynucleotide and said target polynucleotide outside said target sequence.

13. The method according to claim 11 wherein probe P_{A} and probe P_{B} are differently labelled.

14. A computer program product for performing, when executed on a computing device, a method for determining the presence of a mutant T_{M} of a target polynucleotide T_{wt} in a sample solution according to claim 1, said computer program product being configured for receiving hybridisation intensity I(P_{A})m for a sample solution with a first probe P_{A};
receiving hybridisation intensity I(P_{B})m for a sample solution with a second probe P_{B};
analysing and comparing I(P_{A})m and I(P_{B})m, preferably determining the ratio I(P_{A})m/I(P_{B})m, and determining the presence of a mutant polynucleotide in said sample solution based thereon.

15. A device configured for performing the method for determining the presence of a mutant of a target polynucleotide in a sample solution according to claim 1, comprising one or more sets of reaction vessels, feeds for reagents connected thereto and a detection unit and a processing unit comprising the computer program product according to claim 14.

16. The method according to claim 2, wherein said first probe P_{A} and second probe P_{B} are selected so that the ratio of the hybridisation intensity [I(P_{A})] for the hybridisation between the target polynucleotide T_{wt} and the first probe P_{A} to the hybridisation intensity [I(P_{B})] for the hybridisation between the target polynucleotide T_{wt} and the second probe P_{B} is known and ranges between between 0.1 and 10.


SEQUENCE LISTING (7 sequences)

SEQ ID NO: 1 - DNA, 222 bp, Homo sapiens
gtcctgcaccagtaatatgcatattaaaacaagatttacctctattgttggatcatattcgtccacaaaatgattctgaattagctgtatcgtcaaggcactcttgcctacgccaccagctccaactaccacaagtttatattcagtcattttcagcaggccttataataaaaataatgaaaatgtgactatattagaacatgtcacacataaggttaatac

SEQ ID NO: 2 - DNA, 222 bp, Homo sapiens
gtcctgcaccagtaatatgcatattaaaacaagatttacctctattgttggatcatattcgtccacaaaatgattctgaattagctgtatcgtcaaggcactcttgcctacgccagcagctccaactaccacaagtttatattcagtcattttcagcaggccttataataaaaataatgaaaatgtgactatattagaacatgtcacacataaggttaatac

SEQ ID NO: 3 - DNA, 23 bp, Artificial Sequence
  Perfect match probe PT
gttggagctggtggcgtaggcaa

SEQ ID NO: 4 - DNA, 23 bp, Artificial Sequence
  Mutant match probe PA
gttggagctgctggcgtaggcaa

SEQ ID NO: 5 - DNA, 23 bp, Artificial Sequence
  “Insensitive” probe PB
gttggggctggtggcgaaggcaa

SEQ ID NO: 6 - DNA, 23 bp, Artificial Sequence
  Mutant probe PA
gttggagctgctggcggaggcaa

SEQ ID NO: 7 - DNA, 23 bp, Artificial Sequence
  “Insensitive” probe PB
gttggagttggtggcgtcggcaa